(19) United States
(12) Patent Application Publication
(10) Pub. No.: US 2026/0232220 A1
(43) Pub. Date: Aug. 13, 2026

(54) SYSTEM AND METHOD FOR DETERMINING NAVICULAR DROP MEASUREMENTS

(71) Applicant: XRPro, LLC, Boulder, CO (US)

(72) Inventors: Kazi Miftahul Hoque, Jashore (BD); Ravi Vibhakar Shah, Boulder, CO (US); Dmitrii Aleksandrovich Gladyshev, Longmont, CO (US)

(21) Appl. No.: 19/156,517

(22) PCT Filed: Mar. 28, 2024
(86) PCT No.: PCT/US2024/021911

§ 371 Date: Aug. 14, 2025

Related U.S. Application Data
(60) Provisional application No. 63/458,518, filed on Apr. 11, 2023.

(51) Int. Cl. A61B 5/107; A61B 5/00

(52) U.S. Cl. CPC A61B 5/1074; A61B 5/0035; A61B 5/1079; A61B 5/7267; A61B 2576/02

(57) ABSTRACT

A system for determining metrics and conditions of features of a limb of a patient utilizing a three-dimensional sensor data of the limb in a first and a second load bearing state. For example, the system may determine conditions of the limb based on difference or changes in identified metrics when the limb is in the first state of load bearing and the second state of load bearing.

### CROSS-REFERENCE TO RELATED APPLICATIONS

This application is a U.S. national stage application under 35 USC § 371 of International Application No. PCT/US24/21911 filed on Mar. 28, 2024 and entitled “SYSTEM AND METHOD FOR DETERMINING NAVICULAR DROP MEASUREMENTS,” which claims priority to U.S. Provisional Application No. 63/458,518 filed on Apr. 11, 2023 and entitled “SYSTEM AND METHOD CAPABLE OF PERFORMING AUTOMATED NAVICULAR DROP MEASUREMENTS,” which are incorporated herein by reference in their entirety.

### BACKGROUND

Navicular drop is a measure of the difference in arch height of the foot between a relaxed state and when bearing weight. The navicular drop is often an indicator of certain foot conditions such as overpronation, which may lead to pain and injury. Currently, navicular drop is typically determined by manual measurements, which are often subjective and prone to error resulting, in many cases, in misdiagnosis.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is an example block diagram of an architecture for a cloud-based anatomical data processing system according to some implementations.

FIG. 2 is an example process flow diagram associated with determining metrics of a feature of a patient according to some implementations.

FIG. 3 is another example process flow diagram associated with determining metrics of a foot according to some implementations.

FIG. 4 is another example process flow diagram associated with determining a navicular drop of a foot according to some implementations.

FIG. 5 is an example block diagram of an architecture for a cloud-based anatomical data processing system according to some implementations.

FIG. 6 is an example block diagram of an architecture for user equipment associated with an anatomical data processing system according to some implementations.

FIG. 7 is an example pictorial diagram of a three-dimensional model of a foot of a patient according to some implementations.

FIG. 8 is an example pictorial diagram of a three-dimensional model of a foot of a patient according to some implementations.

FIG. 9 is an example pictorial diagram of a two-dimensional footprint showing contact points and non-contact points according to some implementations.

FIG. 10 is an example pictorial diagram of a two-dimensional footprint generated by the anatomical data processing system according to some implementations.

FIG. 11 is an example pictorial diagram of a three-dimensional model of a foot of a patient in a load bearing state according to some implementations.

FIG. 12 is an example pictorial diagram of a three-dimensional model of a foot of a patient in a non-load bearing state according to some implementations.

FIG. 13 is an example pictorial diagram of a three-dimensional model of a foot of a patient showing various additional metrics that may be determined by an anatomical data processing system according to some implementations.

FIG. 14 is an example pictorial diagram of a three-dimensional model of a foot of a patient showing various additional metrics that may be determined by an anatomical data processing system according to some implementations.

### DETAILED DESCRIPTION

Discussed herein are systems and architecture for the processing of three-dimensional (3D) data (such as 3D medical data, healthcare data, image data, and/or the like), in particular 3D health care data of a foot of a patient, for determining a navicular drop and diagnosing associated conditions. For example, the system and processes discussed herein may receive the 3D data representing the foot of the patient in a first position (such as at rest or in a non-load bearing position) and the same foot in a second position (such as standing or in a load bearing position). The system may be configured to determine the height of the arch in both the load bearing and non-load bearing positions and using the determined arch heights in both conditions to determine the navicular drop of the foot.

In some cases, the system may include a user device or equipment equipped with one or more image or sensor devices that may capture the 3D data of the foot in both the load bearing and non-load bearing position. The 3D data of the foot in both positions may be transmitted to a local display device which may in some cases be incorporated into the scanning device. In some instances, the display may provide user data or instructions to assist a healthcare professional, the patient, and/or another user assisting the patient in capturing each of the scans. For example, the instructions may include images with missing portions, directions on where to scan, directions on how the patient should stand, instructions to re-scan portion of the foot, and the like. In some cases, the instructions may be interactive such as displaying a 3D mesh or model of the foot which highlighted or otherwise indicated areas for additional scanning. The display device and/or the user device (herein, the user equipment) may be communicatively coupled to one or more cloud-based services or resources. In some cases, the cloud-based resources may be configured to generate the mesh, model, instructions and or otherwise process the 3D data scanned or produced by the user equipment. For example, the cloud-based resources may determine if a scan is complete or additional data is required. In other cases, the cloud-based resources may determine if the patient is in the correct position for each scan or if there are other inconsistencies or issues that may result in an improper diagnosis or measurement. The system may provide this feedback to the healthcare professional, patient, or other user via the display device.

In some cases, the system, such as the cloud-based resources, may be configured to receive the first 3D data of the foot in a non-load bearing state and second 3D data of the same foot in a load bearing state. For the non-load bearing state, the system may determine a 3D mesh of the foot that may include the ground plane. The system may determine a ground plane in the 3D data, for example, using a first technique (such as a use of a pseudo-inverse of a matrix representing the distance from the N points and an arbitrary plane to find the plane with the smallest Euclidean distance to all points, a Single Value Decomposition (SVD), Random Sample Consensus (RANSAC) technique, a combination technique, or the like). Once the ground plane is determined, the system may filter mesh points of the 3D mesh or model that are less than or equal a threshold distance (such as 1.0 centimeter (cm), 1.5 cm, or between the range of 1.0 to 1.5 cm) from the detected plane. The remaining points are then projected onto the ground plane to determine a two-dimensional (2D) footprint of the patient in the non-load bearing state.

Using the footprint, the system may determine the convex hull of the foot and, based on the convex hull in the non-load bearing state, the system may determine a medial border line (MBL) of the foot in the non-load bearing state. The system may then project the MBL as a line or wall perpendicular with the ground plane, into the 3D model or mesh of the foot and determine an intersection between the 3D mesh or model and the projected line or wall. The system may also determine a mid-point of the MBL based on a length of the MBL and/or the foot of the patient in the non-load bearing state. The system may then measure or determine the distance between the intersection of the 3D mesh or model and the projected line and the ground plane at the mid-point of the MBL. This determined distance may be considered as the first height of the arch or the height of the arch in the non-load bearing state.

For the load bearing state, the system may determine a 3D mesh of the foot that may include the ground plane. The system may determine a ground plane in the 3D data, for example, using a second technique (such as RANSAC technique). Once the ground plane is determined, the system may filter mesh points of the 3D mesh or model that are less than or equal to the threshold distance (such as 1.0 cm, 1.5 cm, or between the range of 1.0 to 1.5 cm) from the detected plane. In the current example, the threshold distance may be the same for the non-load bearing and load bearing model but it should be understood that in other examples, the threshold distances may vary based on the state of the foot when the 3D data is captured. The remaining points are then projected onto the ground plane to determine a 2D footprint of the patient in the load bearing state.

Using the footprint, the system may determine the convex hull of the foot and, based on the convex hull, the system may determine the MBL of the foot in the load bearing state. The system may then project the MBL as a line or wall perpendicular with the ground plane, into the 3D model or mesh of the foot and determine an intersection between the 3D mesh or model and the projected line or wall. The system may also determine a mid-point of the MBL based on a length of the MBL and/or the foot of the patient in the load bearing state. The system may then measure or determine the distance between the intersection of the 3D mesh or model and the projected line and the ground plane at the mid-point of the MBL. This determined distance may be considered as the second height of the arch or the height of the arch in the load bearing state.

Once both the first and second heights of the arch of the foot are determined, the system may determine a difference between the first height and the second height. The difference between the first height and the second height may be the navicular drop of the foot. The system may then process the first height of the foot in the non-load bearing state, the second height of the foot in the load bearing state, and/or the navicular drop of the foot to diagnose foot conditions, to notify a health care professional to further investigate concerns, or to recommend additional diagnostic scans of the foot. The system may also output to a system associated with the healthcare professional, the patient, the user equipment, or another user associated with the patient (such as a secondary health care professional, third-party, or the like) the first height of the foot in the non-load bearing state, the second height of the foot in the load bearing state, and/or the navicular drop. In some cases, the system may repeat the process for the second foot of the patient such that a navicular drop of each foot may be determined for the patient.

In some cases, the 3D data may be processed via cloud based services, systems, and/or processing resources. In some examples, the cloud-based services may include, among other elements, one or more servers in communication with one or more user equipment or devices over one or more networks. In other cases, the 3D data may be processed locally on a user equipment or partially in the cloud and partially on the local user equipment.

In some implementations, the 3D data may include numeric representations of anatomy of patients or users, such as three-dimensional scans of body parts (e.g., the foot of the patient), portions of skin, organs (internal or external), and the like. The 3D data may also include different types of data, such as thermal data, red-green-blue data, depth data, infrared data, Magnetic resonance imaging (MRI) data, light detection and ranging (LIDAR) data, and the like. In some cases, the 3D data may also include additional data related to the image data such as sensor data including one or more of temperature, oxygenation, bacterial load, electrical potential, dielectric impedance, electrocardiogam (EKG), photoplethysmograph (PPG), heart rate, heart rate variance (HRV), and the like. In some cases, meta-data may be associated with the 3D data. For instance, the meta data may include patient information (e.g., identifiers, demographic information, name, age, gender, weight, body part dimensions, such as extracted from the 3D data by the capture device, birth date, medical history, family data, and the like), 3D scan or scanning device information (e.g., device identifier, sensor type, serial number, firmware or software version, scan date, time, and/or the like).

In some case, as discussed herein, cloud-based processing may consist of multiple servers, available full-time and on demand, making their services substantially ubiquitous, constantly available, on demand and easily accessible (e.g., additional servers may be quickly activated in times of peak demand). Also, servers may consist of multiple computers in large service centers, using different operating systems, benefiting from lower-cost centralized utilities and services, and from expandable infrastructure, such as multiple parallel central processing units (CPUs), graphic processing units (GPUs), arithmetic logic units (ALUs), tensor processing units (TPUs) and quantum processing units (QPUs), to name a few. In general, cloud-servers may also be referred to as backend-servers or simply backend.

In some implementations, the cloud-based system discussed herein may include data pre-processing, use of one or more machine learning models that are trained on 3D data associated with anatomy of individuals having various conditions, symptoms, states of health, age, genders, cultural backgrounds, and the like. For example, the one or more machine learning models may be trained to segment the 3D data, classify the 3D data, perform feature detection (such as identifying body parts, landmarks, dimensions, and the like) from the 3D data. The one or more machine learning models may also be trained to assist in diagnosing conditions and symptoms with respect to various body parts and individuals having a wide variety of features, body types, lifestyles (e.g., diet, exercise, work conditions, and the like), cultures, demographics (e.g., age, gender, and the like), such as those classified and identified by one or more other machine learning models (including, but not limited to, feet in various stages of load bearing and having various types and conditions of arches), determining health status, recommending patient specific treatments or therapies and the like.

In this manner, a health care professional may upload 3D data via the user equipment to the cloud-based system and receive in response indications of body parts or conditions that may require further evaluation, user specific anthropometric measurements to assist with diagnostics or evaluations, flagged or identified potential conditions, symptoms as well as suggested treatments or therapies including those related to navicular drops in the arch of the foot. In some cases, the cloud-based system may provide instructions to perform additional scans and/or capture additional 3D data associated with a specific user to enhance any recommendations or features identified. In one specific example, the cloud-based system may also return one or more additional inquiries for the healthcare professional and/or the patient, such as questions related to an accident, a particular body part, history of a body part, feature, or change in state detected, and the like to further assist the healthcare professionals in diagnostics and evaluation of the patient.

As described herein, the machine learning models may be generated using various machine learning techniques. For example, the models may be generated using one or more neural network(s). A neural network may be a biologically inspired algorithm or technique which passes input data (e.g., image and sensor data captured by the user equipment or devices) through a series of connected layers to produce an output or learned inference. Each layer in a neural network can also comprise another neural network or can comprise any number of layers (whether convolutional or not). As can be understood in the context of this disclosure, a neural network can utilize machine learning, which can refer to a broad class of such techniques in which an output is generated based on learned parameters.

As an illustrative example, one or more neural network(s) may generate any number of learned inferences or heads from the captured sensor and/or image data. In some cases, the neural network may be a trained network architecture that is end-to-end. In one example, the machine learning models may include segmenting and/or classifying extracted deep convolutional features of the sensor and/or image data into semantic data. In some cases, appropriate truth outputs of the model in the form of semantic per-pixel classifications (e.g., vehicle identifier, container identifier, driver identifier, and the like).

Although discussed in the context of neural networks, any type of machine learning can be used consistent with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like. In some cases, the system may also apply Gaussian blurs, Bayes Functions, color analyzing or processing techniques and/or a combination thereof.

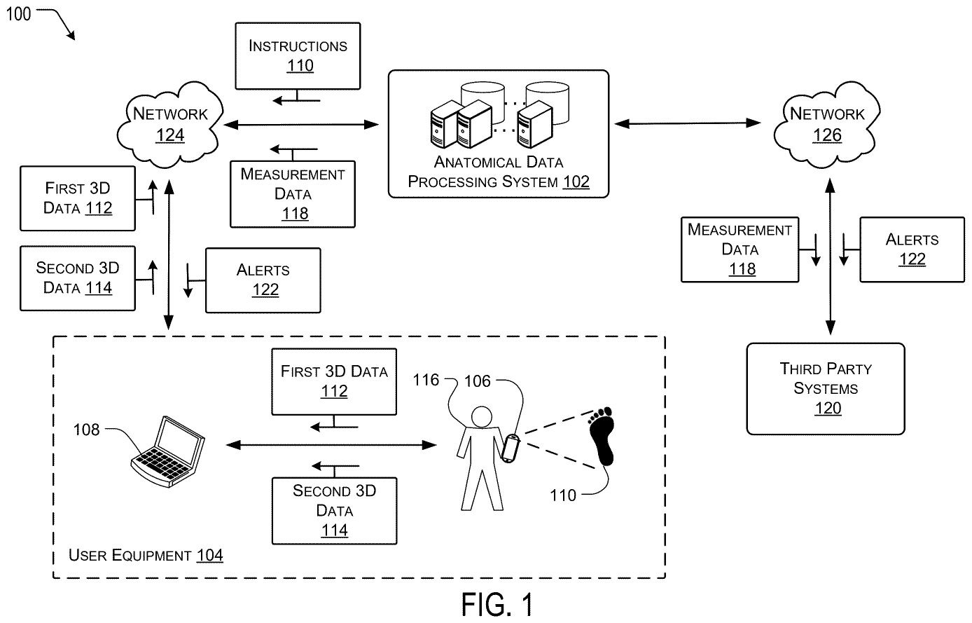

FIG. 1 is an example block diagram of an architecture 100 for a cloud-based anatomical data processing system 102 according to some implementations. In the current example, user equipment 104, such as a sensor device 106 and a processing device 108, may be used to generate first 3D data 112 representative of a foot 110 of a patient in a first state (such as at rest or in a non-load bearing state) and second 3D data 114 of the foot 110 in a second state (such as standing or in a load bearing state).

In the current example, the sensor device 106 may capture the first 3D data 112 and the second 3D data 114 and provide the data 112 and 114 to the processing device 108. The processing device 108 may generate a first mesh associated with the first 3D data 112 and a second mesh associated with the second 3D data 114 that may be displayed on a display of the processing device 108 to assist with data capture by a user 116. In some implementations, the user equipment 104 may provide the first 3D data 112 and the second 3D data 114 to the cloud-based anatomical data processing system 102. In other examples, the user equipment 104 may provide the generated meshes as a pre-processed 3D data to assist the cloud-based anatomical data processing system 102 in determining the measurement data 118.

In the current example, the sensor device 106 and the processing device 108 of the user equipment 104 are illustrated as separate devices. However, it should be understood that in some cases the user equipment 104 may be a single device for both processing the 3D data and capturing the 3D data. The single user equipment 104 may also include a user interface and display or a combination touch screen interface to both receive user inputs and present data to the user 116 (such as instructions 120 from the cloud-based anatomical data processing system 102, the 3D meshes, the 3D data 112 and/or 114, or the like). For example, the user equipment 104 may be configured to provide user data or instructions to assist the user 116 (e.g., the health care professional, the patient, and/or another user assisting the patient) in capturing each of the scans. For instance, the instructions may include images with missing portions, directions on where to scan, directions on how the patient should stand, instructions to re-scan portion of the foot, and the like. In some cases, the instructions may be interactive such as displaying a 3D mesh or model of the foot 110 which highlighted or otherwise indicated areas for additional scanning.

Once the anatomical data processing system 102 receives the first 3D data 112 of the foot 110 in the first state (e.g., the non-load bearing state) and the second 3D data 114 of the foot 110 in the second state (e.g., the load bearing state), the anatomical data processing system 102 may process the first 3D data 112 and the 3D data 114 individually. In some cases, the processing of the first 3D data 112 and the 3D data 114 may be concurrently (e.g., as parallel processes) in other cases the first 3D data 112 and the 3D data 114 may be processed serially or as partially concurrent operations.

For the first 3D data 112 representative of the foot 110 in the non-load bearing state, the system 102 may determine a 3D mesh of the foot 110 (such as if not provided by the processing device 108) that may include a ground plane. The anatomical data processing system 102 may disambiguate the ground plane in the first 3D data 112 from the foot 110. For example, the anatomical data processing system 102 may use a first technique such as by applying a use of a pseudo-inverse of a matrix representing the distance from the N points and an arbitrary plane to find the plane with the smallest Euclidean distance to all points and assign that plane as the ground plane. Once the ground plane is determined, the anatomical data processing system 102 may filter mesh points of the 3D mesh or model that are less than or equal a threshold distance (such as 1.0 cm, 1.5 cm, or between the range of 1.0 to 1.5 cm) from the detected ground plane. Next, the anatomical data processing system 102 may project the remaining points onto the ground plane to determine a 2D footprint of the patient in the non-load bearing state.

Using the footprint, the anatomical data processing system 102 may determine the convex hull of the foot 110 and, based on the convex hull in the non-load bearing state, the anatomical data processing system 102 may determine the MBL of the foot in the non-load bearing state. The anatomical data processing system 102 may then project the MBL as a line or wall perpendicular with the ground plane, into the 3D model or mesh of the foot 110 and determine an intersection between the 3D mesh or model and the projected line or wall.

The anatomical data processing system 102 may also determine a mid-point of the MBL based on a length of the MBL and/or the foot 110. The anatomical data processing system 102 may then measure or determine the distance between the intersection of the 3D mesh or model and the projected line and the ground plane at the mid-point of the MBL. This determined distance may be considered as the first height of the arch of the foot 110 or the height of the arch of the foot 110 in the non-load bearing state.

For the load bearing state, the anatomical data processing system 102 may, again if needed, determine a 3D mesh of the foot 110 that may include the ground plane. The second model or mesh may be of the foot 110 in the load bearing state. The anatomical data processing system 102 may determine the ground plane in the second 3D data 114, for example, using a second technique (such as RANSAC technique). Once the ground plane is determined, the anatomical data processing system 102 may filter mesh points of the 3D mesh or model that are less than or equal to the threshold distance (such as 1.0 cm, 1.5 cm, or between the range of 1.0 to 1.5 cm) from the detected plane. In the current example, the threshold distance may be the same for the non-load bearing and load bearing model but it should be understood that in other examples, the threshold distances may vary based on the state of the foot 110 when the second 3D data 114 is captured. The anatomical data processing system 102 may then project the remaining points onto the ground plane to determine a second 2D footprint of the patient in the load bearing state.

Using the second 2D footprint, the anatomical data processing system 102 may determine the convex hull of the foot 110 in the load bearing state and, based on the convex hull, the anatomical data processing system 102 may determine the MBL of the foot in the load bearing state. The anatomical data processing system 102 may then project the MBL as a line or wall perpendicular with the ground plane, into the 3D model or mesh of the foot 110 in the load bearing state and determine an intersection between the 3D mesh or model and the projected line or wall.

The anatomical data processing system 102 may also determine a mid-point of the MBL based on a length of the MBL and/or the foot 110 of the patient in the load bearing state. The anatomical data processing system 102 may then measure or determine the distance between the intersection of the 3D mesh or model and the projected line and the ground plane at the mid-point of the MBL. This determined distance may be considered as the second height of the arch of the foot 110 or the height of the arch in the load bearing state.

Once both the first and second heights of the arch of the foot 110 are determined, the anatomical data processing system 102 may determine a difference between the first height and the second height. The difference between the first height and the second height may be the navicular drop of the foot 110. The anatomical data processing system 102 may then process the first height of the foot 110 in the non-load bearing state, the second height of the foot 110 in the load bearing state, and/or the navicular drop of the foot 110 to diagnose foot conditions, to notify a health care professional to further investigate concerns, or to recommend additional diagnostic scans of the foot.

The anatomical data processing system 102 may also output to the user equipment 104 and/or a third-party system 120 (e.g., a healthcare professional system, insurance system, patient portal, and/or the like) the first height of the foot 110 in the non-load bearing state, the second height of the foot 110 in the load bearing state, and/or the navicular drop as the measurement data 118. In some cases, the anatomical data processing system 102 may repeat the process for the second foot of the patient such that a navicular drop of each foot may be determined for the patient and output as measurement data 118.

In some cases, the anatomical data processing system 102 may also determine or diagnose foot conditions. In these cases, the anatomical data processing system 102 may output the conditions, additional instructions, additional examination procedures, treatment data, or the like to the user equipment 104 or a third-party system 120 as alerts 122. As one example, the alerts 122 may be generated by one or more machine learning models. The one or more machine learning models may be trained to assist in diagnosing conditions and symptoms with respect to the foot 110 of the patient by using training data that includes 3D data of feet in different positions, with different conditions present, in different states of load bearing, and of different individuals. The different individuals may have a wide variety of features (such as those classified and identified by one or more other machine learning models), changes in state of the features, determining health status, be undergoing different patient specific treatments or therapies and the like. In this manner, a health care professional may upload 3D data of the foot 110 via a user equipment 104 to the cloud-based system 102 and receive in response indications of conditions that may require further evaluation, user specific anthropometric measurements to assist with diagnostics or evaluations, flagged or identified potential conditions, symptoms as well as suggested treatments or therapies. In some cases, the cloud-based system 102 may provide alerts 122 with instructions to perform additional scans and/or capture additional 3D data associated with a specific patient to enhance any recommendations or features identified. In one specific example, the cloud-based system 102 may also return one or more additional inquiries for the healthcare professional and/or the patient, such as questions related to an accident, a particular body part, history of a feature detected, history of a change in state of a feature detected, and the like to further assist the healthcare professionals in diagnostics and evaluation of the patient.

As discussed above, the system 102 may also utilize one or more machine learning models (that may differ from the diagnostic machine learning models) for determining the measurement data 118 (e.g., the height of the arches and the navicular drop). These one or more machine learning models may also be trained on 3D data of feet of various individuals in various different conditions and states of load bearing. Accordingly, the system 102 may utilize multiple sets of machine learning models, as discussed herein.

In some case, as discussed herein, cloud-based processing may consist of multiple servers, available full-time and on demand, making their services substantially ubiquitous, constantly available, on demand and easily accessible (e.g., additional servers may be quickly activated in times of peak demand). Also, servers may consist of multiple computers in large service centers, using different operating systems, benefiting from lower-cost centralized utilities and services, and from expandable infrastructure, such as multiple parallel central processing units (CPUs), graphic processing units (GPUs), arithmetic logic units (ALUs), tensor processing units (TPUs) and quantum processing units (QPUs), to name a few. In general, cloud-servers may also be referred to as backend-servers or simply backend.

In the current example, the data, alerts, measurements, instructions, and the like may be transmitted between various systems using networks, generally indicated by 124-126. The networks 124-126 may be any type of network that facilitates compunction between one or more systems and may include one or more cellular networks, radio, WiFi networks, short-range or near-field networks, infrared signals, local area networks, wide area networks, the internet, and so forth. In the current example, each network 124-126 is shown as a separate network but it should be understood that two or more of the networks may be combined or the same.

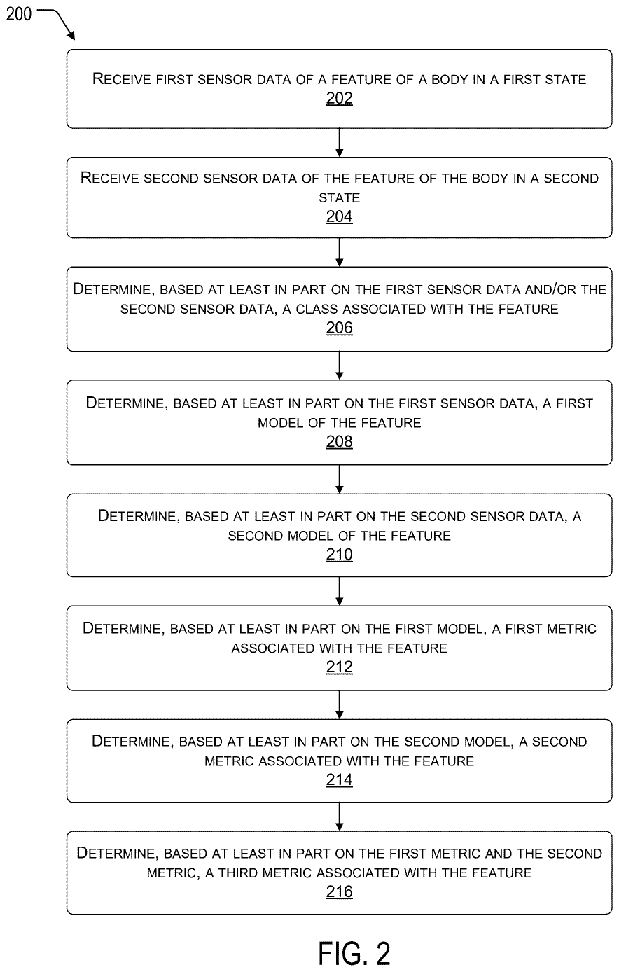

FIGS. 2-4 are flow diagrams illustrating example processes associated with the scheduling system discussed herein. The processes are illustrated as a collection of blocks in a logical flow diagram, which represent a sequence of operations, some or all of which can be implemented in hardware, software, or a combination thereof. In the context of software, the blocks represent computer-executable instructions stored on one or more computer-readable media that, which when executed by one or more processor(s), perform the recited operations. Generally, computer-executable instructions include routines, programs, objects, components, encryption, deciphering, compressing, recording, data structures and the like that perform particular functions or implement particular abstract data types.

The order in which the operations are described should not be construed as a limitation. Any number of the described blocks can be combined in any order and/or in parallel to implement the processes, or alternative processes, and not all of the blocks need be executed. For discussion purposes, the processes herein are described with reference to the frameworks, architectures and environments described in the examples herein, although the processes may be implemented in a wide variety of other frameworks, architectures or environments.

FIG. 2 is an example process flow diagram 200 associated with determining metrics of a feature (or change in state of the feature) of a patient according to some implementations. As discussed above, an anatomical data processing system may assist with determining metrics of a feature (such as a foot, hand, other appendage or the like) on behalf of a medical health professional. For example, in some cases, the metrics may be a distance, size, circumference, change in state, or the like of the feature. In some cases, the system may receive 3D data of the feature in two or more states (such as a load bearing and non-load bearing state) and determine the metrics based at least in part on differences in the 3D data.

At 202, the system may receive first sensor data of a feature (or portion) of a body in a first state and, at 204, the system may receive second sensor data of a feature of a body in a second state. For example, the first sensor data may be first 3D image data of a foot in a load bearing state (e.g., the first state) and the second sensor data may be 3D image data of the foot in a non-load bearing state (e.g., the second state).

At 206, the system may determine, based at least in part on the first sensor data and/or the second sensor data, a class associated with the feature or portion of the human body. For example, the system may determine the feature is a foot or other part of the body. In some cases, the classification may include inputting the first sensor data and the second sensor data into one or more machine learned models trained to classify image data of the body into a class of body part or distinguish between body parts (such as a right and left foot). In some cases, the one or more machine learned models may be trained to classify features of the body based on image data of bodies of individuals having various demographics, life stages, conditions, and in various states, such as standing, sitting, moving, and the like.

At 208, the system may determine, based at least in part on the first sensor data, a first model of the feature (or portion of the human body). For example, using the first sensor data, the system may generate a first mesh or 3D model of the foot. In some cases, the first model may include the ground plane, such as the floor when the model is of a foot.

At 210, the system may determine, based at least in part on the second sensor data, a second model of the feature (or the portion of the human body). For example, using the second sensor data, the system may generate a second mesh or 3D model of the foot. In some cases, the second model may also include the ground plane.

At 212, the system may determine, based at least in part on the first model, a first metric associated with the feature (or the portion of the human body). For example, the system may determine or distinguish the ground plane from the feature (such as the foot). The system may then determine a metric associated with the foot based on the ground plane and the 3D model. For example, the system may determine a first height of an arch of a foot by measuring a distance between the ground plane and the foot at one or more points (such as at the mid-point of the MBL).

At 214, the system may determine, based at least in part on the second model, a second metric associated with the feature(or the portion of the human body). For example, the system may determine or distinguish the ground plane from the feature (such as the foot). The system may then determine a second metric associated with the foot based on the ground plane and the 3D model. For example, the system may determine a second height of an arch of the foot by again measuring a distance between the ground plane and the foot at one or more points (such as at the mid-point of the MBL).

At 216, the system may determine, based at least in part on the first metric and the second metric, a third metric associated with the feature (or the portion of the human body). For example, when the feature is a foot and the first and second metrics are arch heights, the system may determine a navicular drop or difference between the arch height when the foot is load bearing and non-load bearing. In some cases, the third metric may represent a change in state of the feature (or the portion of the human body) when the feature is in a load bearing state and when the feature is in a non-load bearing state.

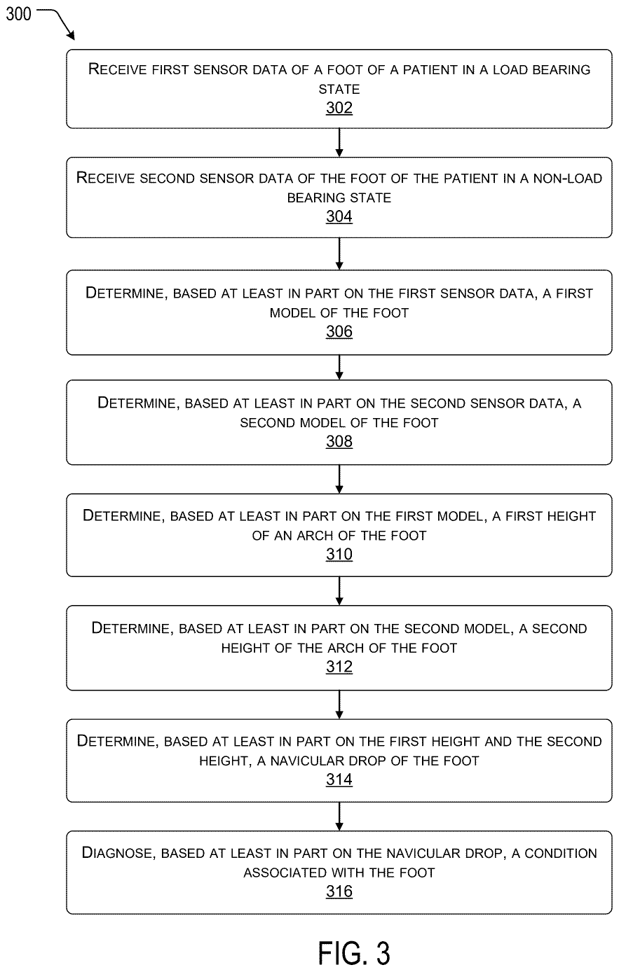

FIG. 3 is another example process flow diagram 300 associated with determining metrics of a foot according to some implementations. As discussed above, an anatomical data processing system may assist with determining metrics of a feature (such as a foot, hand, other appendage or the like) on behalf of a medical health professional. In some cases, the system may receive 3D data of the feature in two or more states (such as a load bearing and non-load bearing state) and determine the metrics based at least in part on differences in the 3D data.

At 302, the system may receive first sensor data of a foot of a patient in a load bearing state and, at 304, the system may receive second sensor data of the foot of a patient in a non-load bearing state. For example, a health care professional or user may capture sensor data of a left foot of the patient while the patient is standing on their left foot then again while the patient is standing on their right foot.

At 306, the system may determine, based at least in part on the first sensor data, a first model of the foot. For example, using the first sensor data, the system may generate a first mesh or 3D model of the foot. In some cases, the first model may include the ground plane, such as the floor when the model is of a foot.

At 308, the system may determine, based at least in part on the second sensor data, a second model of the foot. For example, using the second sensor data, the system may generate a second mesh or 3D model of the foot. In some cases, the second model may also include the ground plane.

At 310, the system may determine, based at least in part on the first model, a first height of the arch of the foot. For example, the system may determine or distinguish the ground plane from the feature (such as the foot). The system may then determine a first height associated with arch of the foot based on the ground plane and the 3D model.

At 312, the system may determine, based at least in part on the second model, a second height of the arch of the foot. For example, the system may again determine or distinguish the ground plane from the feature (such as the foot). The system may then determine a second height associated with the arch of the foot based on the ground plane and the 3D model.

At 314, the system may determine, based at least in part on the first height and the second height, a navicular drop associated with the foot and, at 316, the system may diagnose, based at least in part on the navicular drop, a condition associated with the foot. For example, the system may determine a difference between the arch height when the foot is load bearing and non-load bearing.

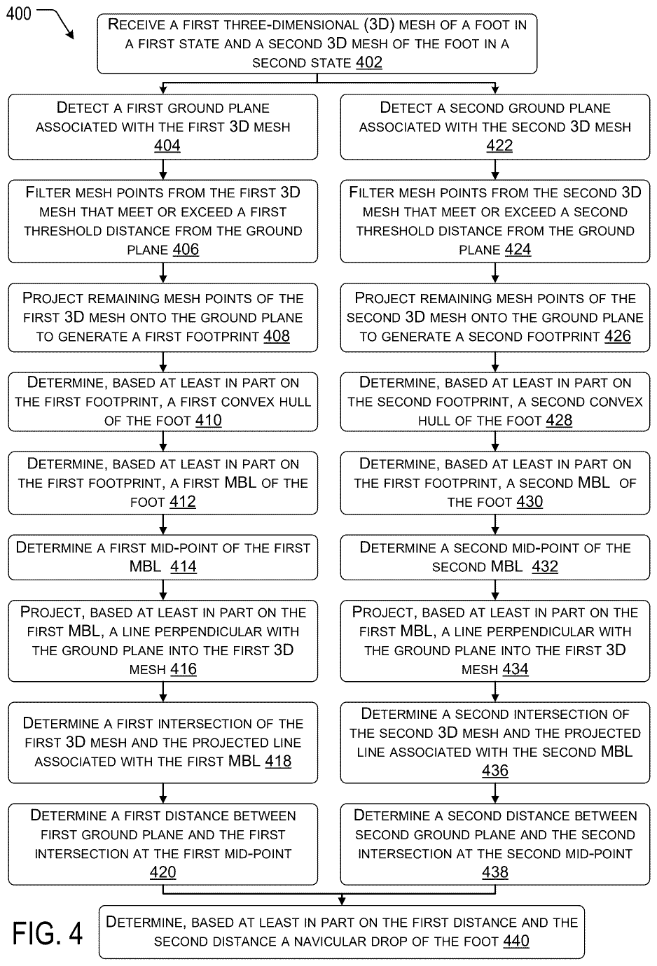

FIG. 4 is another example process flow diagram 400 associated with determining a navicular drop of a foot according to some implementations. As discussed above, an anatomical data processing system may assist with determining metrics of a foot on behalf of a medical health professional. In some cases, the system may receive 3D data or meshes of the foot in two or more states (such as a load bearing and non-load bearing state) and determine the metrics based at least in part on differences in the 3D data or meshes.

At 402, the system may receive a first three-dimensional (3D) mesh of a foot in a first state and a second 3D mesh of the foot in a second state. As discussed above, the first state may be a load bearing state and the second state may be a non-load bearing state. For example, the first 3D mesh and the second 3D mesh may be generated by the user equipment such that as a user, such as a health care professional, scans the foot of a patient, the 3D mesh is generated and concurrently displayed to the user. In this manner, the 3D mesh may assist in providing a higher quality scan that better represents the full features of the foot.

In other examples, the system may be configured to receive the first 3D data of the foot in the first state (e.g., a non-load bearing state) and second 3D data of the same foot in a second sate (e.g., the load bearing state). In these examples, the cloud-based anatomical data processing system may generate the first and second 3D meshes. In some cases, the system may provide the meshes back to the user equipment such that the 3D meshes can be displayed to the user while the user captured the sensor data used to generate each mesh.

At 404, the system may detect a first ground plane associated with the first 3D mesh (e.g., the mesh representing the foot in the non-load bearing state). For example, using a first technique (such as a use of a pseudo-inverse of a matrix representing the distance from the N points and an arbitrary plane to find the plane with the smallest Euclidean distance to all points, a Single Value Decomposition (SVD), Random Sample Consensus (RANSAC) technique, a combination technique, or the like) the ground plane may be distinguished from the remainder of the first 3D mesh.

At 406, the system may filter mesh points from the first 3D mesh that meet or exceed a first threshold distance from the ground plane. For example, the threshold distance may be 1.0 cm, 1.5 cm, or between the range of 1.0 to 1.5 cm.

At 408, the system may project remaining mesh points of the first 3D mesh onto the ground plane to generate a first footprint. For example, the projected points may form a first 2D footprint of the foot in the non-load bearing state.

At 410, the system may determine, based at least in part on the first footprint, a first convex hull of the foot in the non-load bearing state and, at 412, the system may determine, based at least in part on the first footprint, a first MBL of the foot in the non-load bearing state. Next, at 414, the system may determine a first mid-point of the first MBL. For example, the system may determine the first MBL by determining a longest line connecting points surrounding an inner arch of the foot.

At 416, the system may project, based at least in part on the first MBL, a line perpendicular with the ground plane into the first 3D mesh. For example, the line may be a vertical wall or 2D surface that is projected upward from the ground plane based on the first MBL. Next, at 418, the system may determine a first intersection of the first 3D mesh and the projected line associated with the first MBL. For example, the intersection may be a set of points at which the vertical projection upward from the ground plane intersects the first 3D mesh of the foot.

At 420, the system may determine a first distance between the first ground plane and the first intersection at the first mid-point. For example, the system may measure a distance (such as a pixel distance) between the ground plane at the first mid-point upward until the intersection point corresponding to the first mid-point is reached.

At 422, the system may detect a second ground plane associated with the second 3D mesh (e.g., the mesh representing the foot in the load bearing state). For example, using a second technique (such as RANSAC) the ground plane may be distinguished from the remainder of the first 3D mesh. In some cases, the second technique may differ from the first technique.

At 424, the system may filter mesh points from the second 3D mesh that meet or exceed a second threshold distance from the ground plane. For example, the second threshold distance may be 1.0 cm, 1.5 cm, or between the range of 1.0 to 1.5 cm. In various implementations, the second threshold distance may be the same as or differ from the first threshold distance.

At 426, the system may project remaining mesh points of the second 3D mesh onto the ground plane to generate a second footprint. For example, the projected points may form a second 2D footprint of the foot in the load bearing state.

At 428, the system may determine, based at least in part on the second footprint, a second convex hull of the foot in the load bearing state and, at 430, the system may determine, based at least in part on the second footprint, a second MBL of the foot in the load bearing state. Next, at 432, the system may determine a second mid-point of the second MBL.

At 434, the system may project, based at least in part on the second MBL, a line perpendicular with the ground plane into the second 3D mesh. For example, the line may again be a vertical wall or 2D surface that is projected upward from the ground plane based on the second MBL. Next, at 436, the system may determine a second intersection of the second 3D mesh and the projected line associated with the second MBL. For example, the intersection may be a set of points at which the vertical projection upward from the ground plane intersects the second 3D mesh of the foot.

At 438, the system may determine a second distance between the second ground plane and the second intersection at the second mid-point. For example, the system may measure a distance (such as a pixel distance) between the ground plane at the second mid-point upward until the intersection point corresponding to the second mid-point is reached.

At 440, the system may determine, based at least in part on the first distance and the second distance, a navicular drop of the foot. For example, the difference in the first distance and the second distance may be assigned as the navicular drop of the foot.

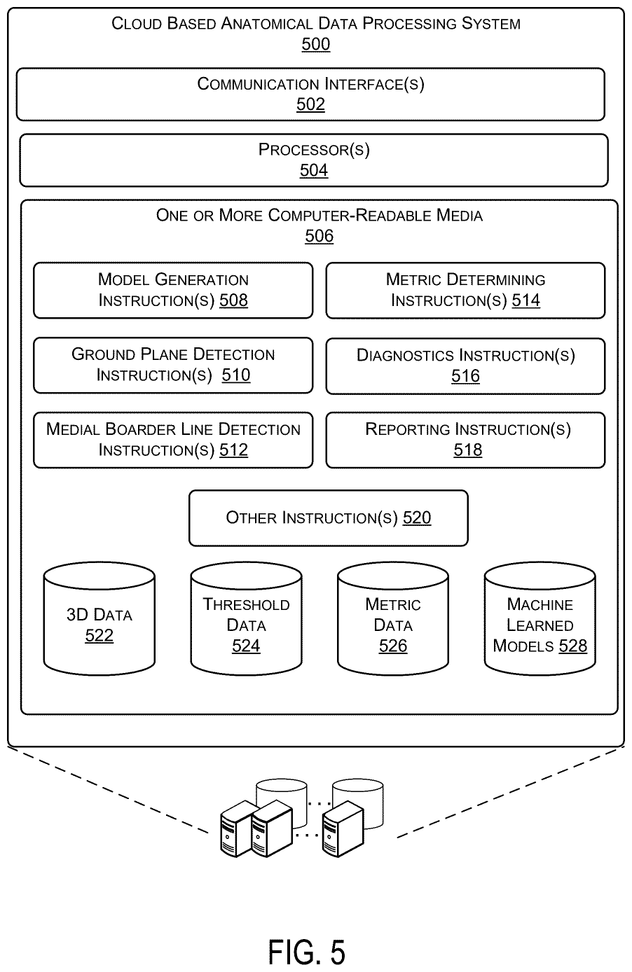

FIG. 5 is an example block diagram of an architecture for a cloud-based anatomical data processing system 500 according to some implementations. The cloud-based anatomical data processing system 500 can include one or more communication interface(s) 502 that enables communication between the cloud-based anatomical data processing system 500 and one or more other local or remote computing device(s) or remote services, such as the user equipment. For instance, the communication interface(s) 502 can facilitate communication with other proximate sensor systems and/or other facility systems. The communications interfaces(s) 502 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The cloud-based anatomical data processing system 500 may include one or more processors 504 and one or more computer-readable media 506. Each of the processors 504 may itself comprise one or more processors or processing cores. The computer-readable media 506 is illustrated as including memory/storage. The computer-readable media 506 may include volatile media (such as random access memory (RAM)) and/or nonvolatile media (such as read only memory (ROM), Flash memory, optical disks, magnetic disks, and so forth). The computer-readable media 706 may include fixed media (e.g., RAM, ROM, a fixed hard drive, and so on) as well as removable media (e.g., Flash memory, a removable hard drive, an optical disc, and so forth). The computer-readable media 506 may be configured in a variety of other ways as further described below.

Several modules such as instructions, data stores, and so forth may be stored within the computer-readable media 506 and configured to execute on the processors 504. For example, as illustrated, the computer-readable media 506 stores model generation instructions 508, ground plane detection instructions 510, MBL detection instructions 512, metric determining instructions 514, diagnostic instructions 516, reporting instruction 518 as well as other instructions 520, such as an operating system. The computer-readable media 506 may also be configured to store data, such as 3D data 522, threshold data 524, metric data 526 (e.g., arch height and navicular drop data), machine learned models 528, as well as other data.

The model generation instructions 508 may be configured to receive the 3D scan or sensor data of the foot and to generate a 3D model or mesh of the foot. In some cases, as discussed herein, the 3D model may include a ground plane or surface that the patient is standing on.

The ground plane detection instructions 510 may process the 3D model of the foot to determine or detect the ground plane. For example, the ground plane detection instructions 510 may disambiguate pixels representing the foot from pixels representing the ground plane.

The MBL detection instructions 512 may be configured to generate a 2D footprint and then detect the MBL based on the 2D footprint. For example, the MBL detection instructions 512 may utilize one or more thresholds, the ground plane, and the 3D model to determine the 2D footprint. The MBL detection instructions 512 may also be configured to detect a mid-point of the MBL with respect to the foot for assisting in determining arch height and navicular drop.

The metric determining instructions 514 may be configured to determine the arch height and the navicular drop of the foot using two models, as discussed herein. For example, the metric determining instructions 514 may utilize a first 3D model of the foot in a load bearing state and a second 3D model of the foot in a non-load bearing state to determine the navicular drop of the foot.

The diagnostic instructions 516 may be configured to detect possible concerns, issues, or conditions with the foot based at least in part on the metrics output by the metric determining instructions 514, such as the arch height in various states of load bearing and/or the navicular drop among others.

The reporting instruction 518 may be configured to send or transmit the diagnostic outputs, the metrics, the 3D models, and the like to one or more third-party system and/or the user equipment responsible for transmitting the 3D sensor data. For example, the reporting instruction 518 may send the diagnostic outputs, the metrics, the 3D models, and the like to insurance providers, medical health care professional systems, patient portals or system, and the like.

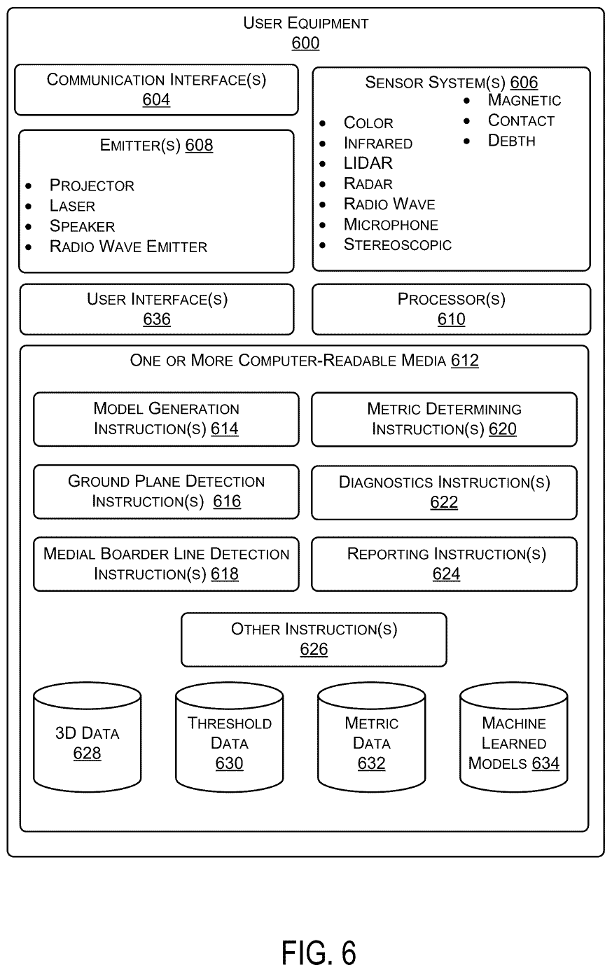

FIG. 6 is an example block diagram of an architecture for user equipment 600 associated with an anatomical data processing system according to some implementations. The user equipment 600 may include one or more communication interface(s) 604 (also referred to as communication devices and/or modems), one or more sensor system(s) 606, and one or more emitter(s) 608.

The user equipment 600 can include one or more communication interfaces(s) 604 that enable communication between the user equipment 600 and one or more other local or remote computing device(s) or remote services, such as a cloud-based system of FIGS. 1-5. For instance, the communication interface(s) 604 can facilitate communication with other proximate sensor systems, a central control system, or other facility systems. The communications interfaces(s) 604 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The one or more sensor system(s) 606 may be configured to capture the 3D data 628 (or other image based data) associated with feature (e.g., a foot or the like) of a patient. In at least some examples, the sensor system(s) 606 may include thermal sensors, time-of-flight sensors, location sensors, LIDAR sensors, radar sensors, sonar sensors, infrared sensors, cameras (e.g., RGB, IR, intensity, depth, etc.), magnetic sensors, microphone sensors, environmental sensors (e.g., temperature sensors, humidity sensors, light sensors, pressure sensors, etc.), and the like. In some examples, the sensor system(s) 606 may include multiple instances of each type of sensor. For instance, camera sensors may include multiple cameras disposed at various locations.

The user equipment 600 may also include one or more emitter(s) 608 for emitting light and/or sound. By way of example and not limitation, the emitters in this example include light, illuminators, lasers, patterns, such as an array of light, audio emitters, and the like.

The user equipment 600 may also include one or more user interfaces 636, such as input (e.g., mouse or keyboard) or output devices (e.g., a display). The user interfaces 636 may include a virtual environment display or a traditional two-dimensional display, such as a liquid crystal display or a light emitting diode display. The user interfaces 636 may also include one or more input components for receiving feedback from the user. In some cases, the input components may include tactile input components, audio input components, or other natural language processing components. In one specific example, the user interfaces 636 may be a combined touch enabled display.

The user equipment 600 may include one or more processors 610 and one or more computer-readable media 612. Each of the processors 610 may itself comprise one or more processors or processing cores. The computer-readable media 612 is illustrated as including memory/storage. The computer-readable media 612 may include volatile media (such as random access memory (RAM)) and/or nonvolatile media (such as read only memory (ROM), Flash memory, optical disks, magnetic disks, and so forth). The computer-readable media 612 may include fixed media (e.g., RAM, ROM, a fixed hard drive, and so on) as well as removable media (e.g., Flash memory, a removable hard drive, an optical disc, and so forth). The computer-readable media 612 may be configured in a variety of other ways as further described below.

Several modules such as instructions, data stores, and so forth may be stored within the computer-readable media 612 and configured to execute on the processors 610. For example, as illustrated, the computer-readable media 612 store instructions similar to those of the system 500 of FIG. 5 including model generation instructions 614, ground plane detection instructions 616, MBL detection instructions 618, metric determining instructions 620, diagnostics instruction 622, reporting instructions 624, as well as other instructions 626. The computer-readable media 612 may also be configured to store data, such as 3D data 628, threshold data 630, metric data 632, machine learned models 634, and the like.

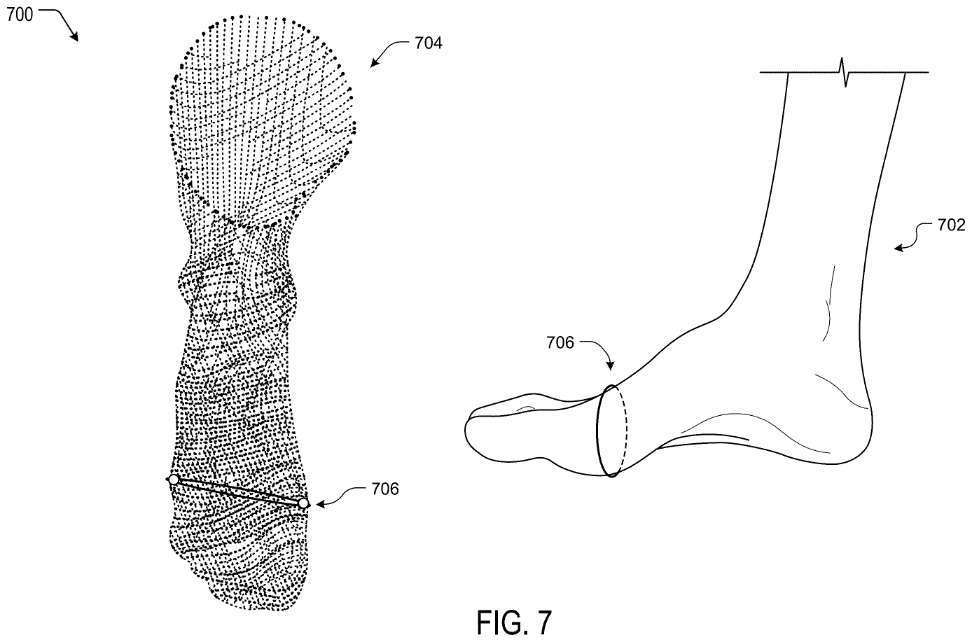

FIG. 7 is an example pictorial diagram of a three-dimensional model 700 of a foot of a patient according to some implementations. In the current example, a 3D model 702 and a 3D mesh 704 is illustrated of a foot. In the current example, a width 706 of the foot is determined as a metric generated by the anatomical data processing system, discussed herein. Accordingly, it should be understood that the system may generate other metrics, such as width, height, length, circumference, diameter at various positions along the foot, and the like.

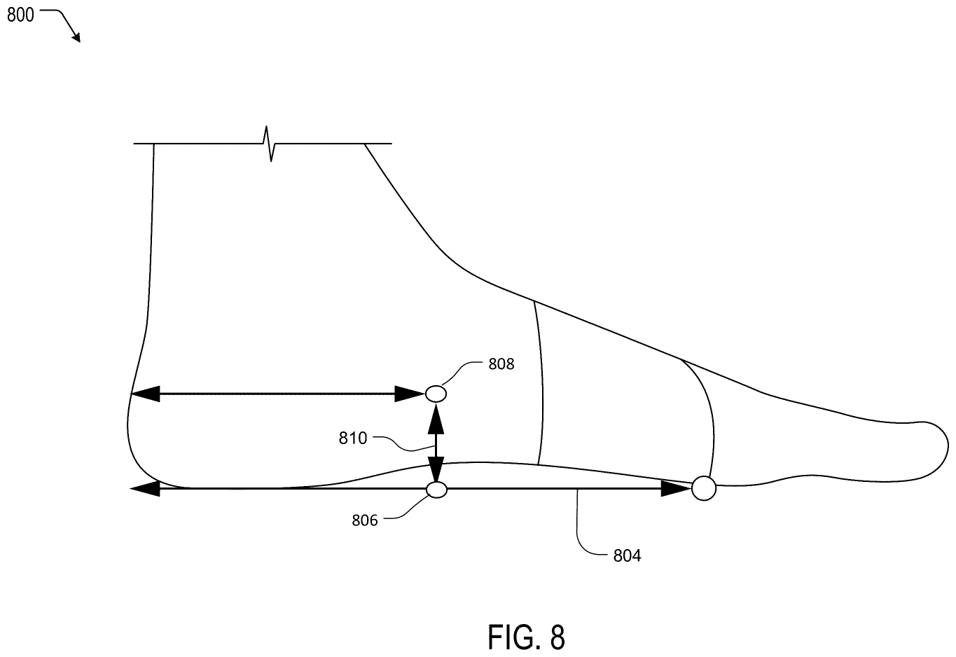

FIG. 8 is an example pictorial diagram of a three-dimensional model 800 of a foot of a patient according to some implementations. In the current example, the anatomical data processing system has determined and placed the MBL of the foot, the ground plane 804, a mid-point 806 of the MBL 802, a projection point 808 of the mid-points onto the model 800 of the foot, and the arch height 810.

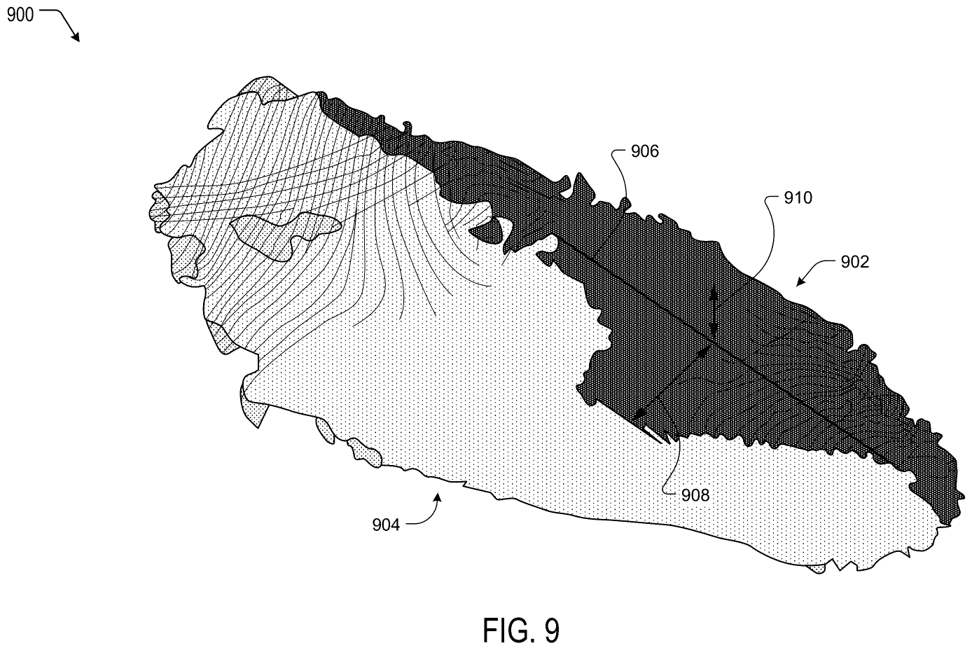

FIG. 9 is an example pictorial diagram of a two-dimensional footprint 900 showing contact points, generally indicated by the light points and 904, and non-contact points, generally indicated by the dark points and 902, according to some implementations. In the current example, the anatomical data processing system may determine the MBL 906, the arch width 908, and the arch height 910.

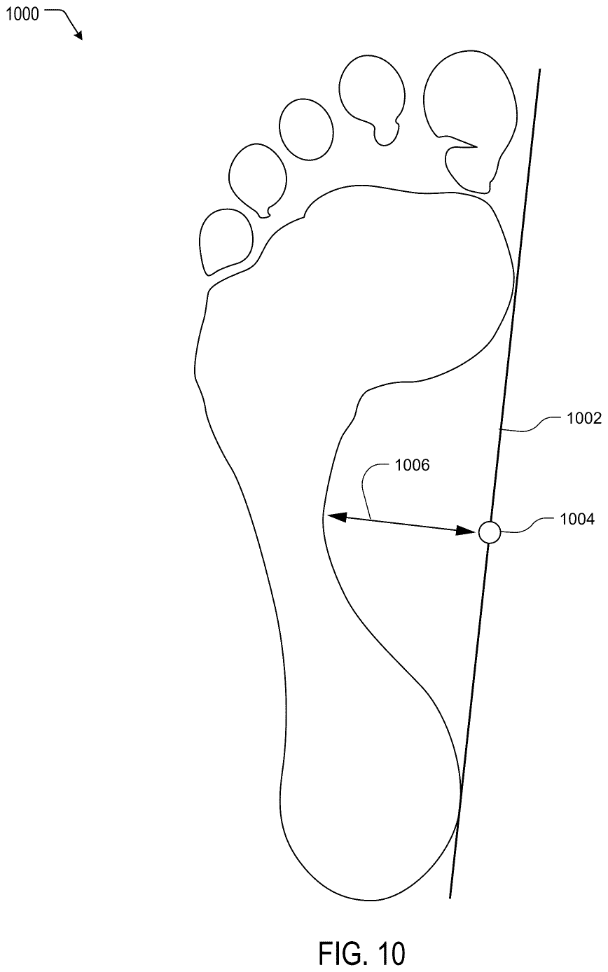

FIG. 10 is an example pictorial diagram of a two-dimensional footprint 1000 generated by the anatomical data processing system according to some implementations. In the current example, the anatomical data processing system has determined the footprint 1000, the MBL 1002, the mid-point of the MBL 1004, and the arch width 1006.

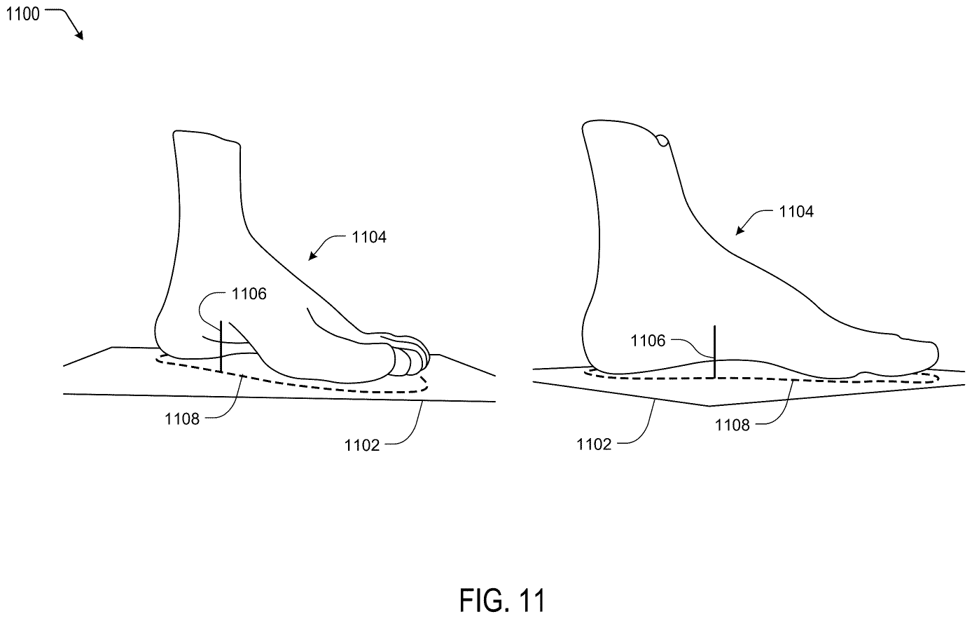

FIG. 11 is an example pictorial diagram of a three-dimensional model 1100 of a foot of a patient in a load bearing state according to some implementations. In the current example, the ground plane 1102 is distinguished from the foot 1104 and the MBL 1106 has been determined, such as based at least in part on the footprint 1108.

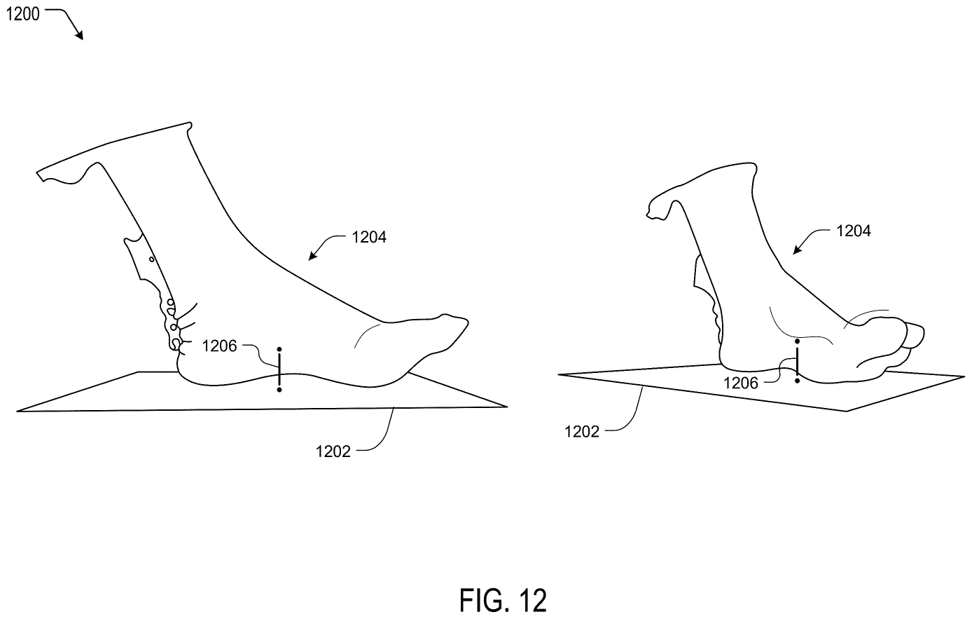

FIG. 12 is an example pictorial diagram of a three-dimensional model 1200 of a foot of a patient in a non-load bearing state according to some implementations. In the current example, the ground plane 1202 is distinguished from the foot 1204 and the MBL 1206 has been determined.

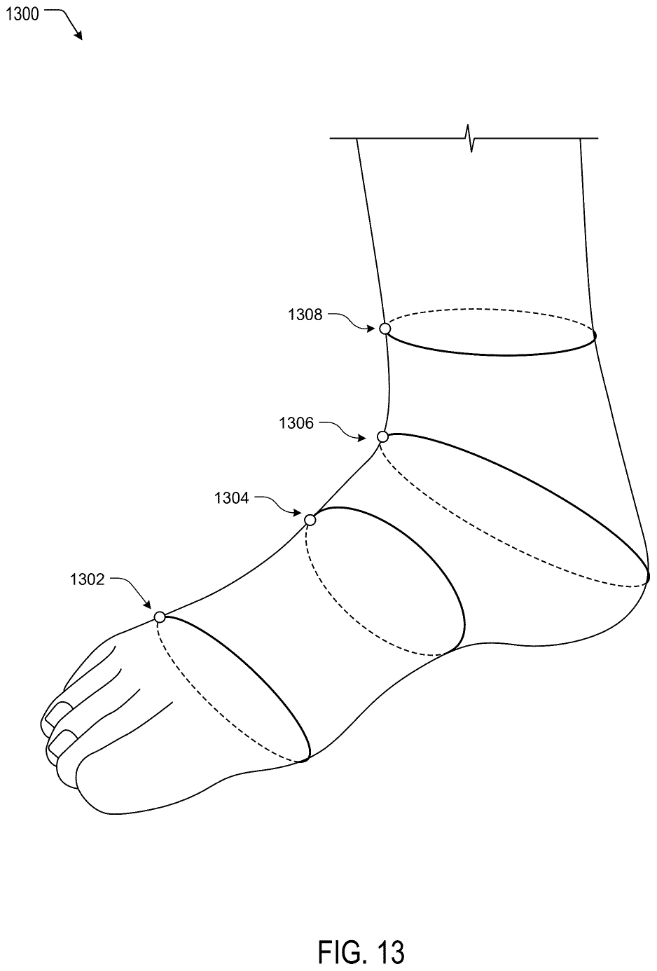

FIG. 13 is an example pictorial diagram of a three-dimensional model 1300 of a foot of a patient showing various additional metrics that may be determined by an anatomical data processing system according to some implementations. For example, the anatomical data processing system may also determine a joint circumference 1302, an instep circumference 1304, a heel circumference 1306, and an ankle circumference 1308.

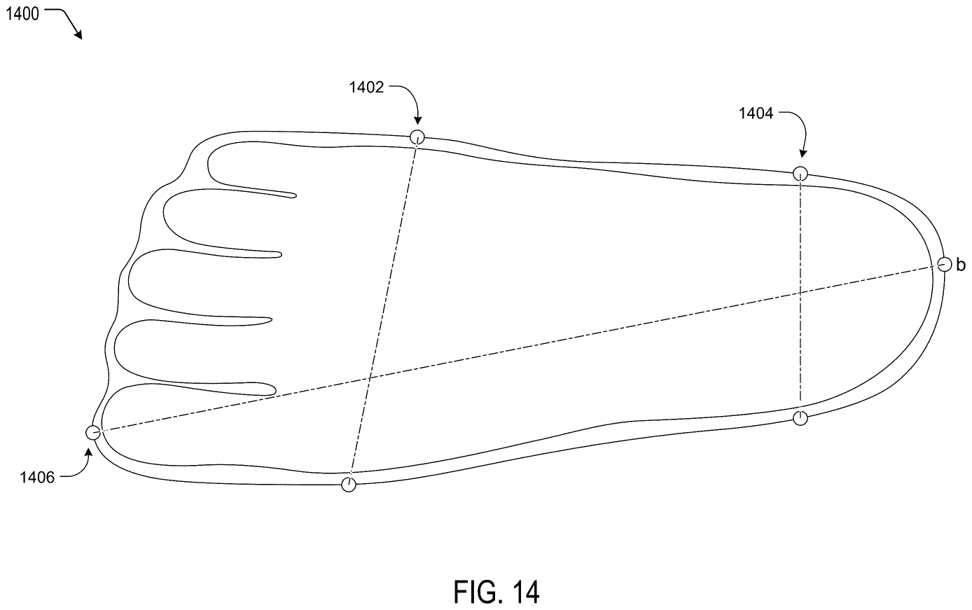

FIG. 14 is an example pictorial diagram of a three-dimensional model 1400 of a foot of a patient showing various additional metrics that may be determined by an anatomical data processing system according to some implementations. For example, the anatomical data processing system may also determine a joint width 1402, a heel width 1404, a foot or orthosis length 1406, and the like.

Although the discussion above sets forth example implementations of the described techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

### EXAMPLE CLAUSES

A. A method comprising: capturing, via an image device associated with a user device, first image data of a portion of a human body in a first state and second image data of the portion of the human body in a second state, the first state different than the second state; determining, based at least in part on the first image data, a first metric associated with the portion of the human body while the portion is in the first state; determining, based at least in part on the second image data, a second metric associated with the portion of the human body while the portion is in the second state; determining, based at least in part on the first metric and the second metric, a third metric associated with the change in state of the portion of the human body; and outputting the third metric.
B. The method of claim A, wherein: the portion of the human body is at least one foot; the first state is in a non-load bearing state; and the second state is in a load bearing state.
C. The method of claim A, further comprising: generating, based at least in part on the first image data, a first three-dimensional model of the portion of the human body; and generating, based at least in part on the second image data, a second three-dimensional model of the portion of the human body.
D. The method of claim A, wherein: the portion of the human body is at least one foot; the first metric is height of an arch of the foot in the non-load bearing state; the second metric is a height of the arch of the foot in the load bearing state; and the third metric is value representative of a navicular drop of an arch of the foot.
E. The method of claim D, further comprising: detecting a first ground plane associated with the first three-dimensional model; filtering mesh points associated with the first three-dimensional model within a threshold distance from the first ground plane to generate a first set of remaining mesh points; determining, based at least in part on the first set of remaining mesh points and the first ground plane, a first footprint associated with the foot in the non-load bearing state; determining, based at least in part on the first three-dimensional model, a first convex hull associated with the foot in the non-load bearing state; and determining, based at least in part on the first footprint and the first convex hull, a medial border line of the foot in the non-load bearing state.
F. The method of claim E, further comprising: detecting a second ground plane associated with the second three-dimensional model; filtering mesh points associated with the second three-dimensional model within the threshold distance from the second ground plane to generate a second set of remaining mesh points; determining, based at least in part on the second set of remaining mesh points and the second ground plane, a second footprint associated with the foot in the load bearing state; determining, based at least in part on the second three-dimensional model, a second convex hull associated with the foot in the load bearing state; and determining, based at least in part on the second footprint and the second convex hull, a medial border line of the foot in the load bearing state.
G. The method of claim F, wherein detecting the first ground plane is associated with a first technique and detecting the second ground plane is associated with a second technique different than the first technique.
H. The method of claim G, wherein: determining the first footprint associated with the foot in the non-load bearing state further comprises projecting the first set of mesh points onto the first ground plane.
I. The method of claim H, wherein determining the second footprint associated with the foot in the load bearing state further comprises projecting the second set of mesh points onto the second ground plane.
J. The method of claim I, further comprising: determining a first intersection point by projecting the medial border line of the foot in the non-load bearing state onto the first three-dimensional model; determining a mid-point of the medial border line of the foot in the non-load bearing state; determining a first distance between the first intersection point and the mid-point of the medial border line of the foot in the non-load bearing state, the first distance being the first metric; determining a second intersection point by projecting the medial border line of the foot in the load bearing state onto the second three-dimensional model; determining a mid-point of the medial border line of the foot in the load bearing state; determining a second distance between the second intersection point and the mid-point of the medial border line of the foot in the load bearing state, the second distance being the second metric; and determining a difference between the first distance and the second distance.
K. The method of claim E, wherein the threshold distance is at least one of the following: approximately 1.5 centimeters; or approximately 1.0 centimeters.
L. The method of claim A, wherein: the portion of the human body is at least one foot; determining the first metric associated with the foot while the foot is in the non-load bearing state is based at least in part on the first three-dimensional model; and determining the second metric associated with the foot while the foot is in the load bearing state is based at least in part on the second three-dimensional model.
M. The method of claim A, wherein the portion of the human body is at least one foot and generating the first three-dimensional model and the second three-dimensional model further comprises: inputting the first image data into one or more machine learned models trained on image data of feet at various load bearing stages and receiving from the one or more machine learned models the first three-dimensional model; and inputting the second image data into the one or more machine learned models and receiving from the one or more machine learned models the second three-dimensional model.
N. The method of claim A, wherein the portion of the human body is at least one foot and determining the first metric, the second metric, or the third metric is further comprises utilizing one or more machine learning models trained on image data of feet in load bearing and non load bearing states of various different individuals having various lifestyles, health status, and demographics.
O. One or more non-transitory computer-readable media storing instructions that, when executed by one or more processors, cause the one or more processors to perform operations comprising: receiving first image data of a foot in a non-load bearing state and second image data of the foot in a load bearing state; determining, based at least in part on the first image data, a first height of an arch of the foot while the foot is in the non-load bearing state; determining, based at least in part on the second image data, a second height of an arch of the foot while the foot is in the load bearing state; determining, based at least in part on the first metric and the second metric, a third metric associated with the arch of the foot; and outputting the third metric.
P. The one or more non-transitory computer-readable media of claim O, wherein the third metric is a navicular drop of the arch of the foot.
Q. A system comprising: one or more processors; and one or more non-transitory computer-readable media storing instructions that, when executed by the one or more processors, cause the one or more processors to perform operations comprising: receiving first sensor data of a foot in a non-load bearing state and second sensor data of the foot in a load bearing state; determining, based at least in part on the first sensor data, a first height of an arch of the foot while the foot is in the non-load bearing state; determining, based at least in part on the second sensor data, a second height of an arch of the foot while the foot is in the load bearing state; determining, based at least in part on the first metric and the second metric, a third metric associated with the arch of the foot; and outputting the third metric.
R. The system of claim Q, wherein the operations further comprise: determining the first metric associated with the arch of the foot is based at least in part on first-three dimensional model generated from the first image data; and determining the second metric associated with the arch of the foot is based at least in part on second-three dimensional model generated from the second image data.
S. The system of claim Q, wherein: the system is a cloud-based system that is remote from a user equipment; and first sensor data and the second sensor data is received from the user equipment.
T. The system of claim Q, wherein the first sensor data and the second sensor data is one or more one of the following:
image data; thermal data; depth data; infrared data; magnetic resonance imaging data, or LIDAR data.

While the example clauses described above are described with respect to one particular implementation, it should be understood that, in the context of this document, the content of the example clauses can also be implemented via a method, device, system, a computer-readable medium, and/or another implementation. Additionally, any of examples A-T may be implemented alone or in combination with any other one or more of the examples A-T.

### CONCLUSION

While one or more examples of the techniques described herein have been described, various alterations, additions, permutations and equivalents thereof are included within the scope of the techniques described herein. As can be understood, the components discussed herein are described as divided for illustrative purposes. However, the operations performed by the various components can be combined or performed in any other component. It should also be understood that components or steps discussed with respect to one example or implementation may be used in conjunction with components or steps of other examples.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

What is claimed is:

1. A method comprising:
capturing, via an image device associated with a user device, first image data of a portion of a human body in a first state and second image data of the portion of the human body in a second state, the first state different than the second state;
determining, based at least in part on the first image data, a first metric associated with the portion of the human body while the portion is in the first state;
determining, based at least in part on the second image data, a second metric associated with the portion of the human body while the portion is in the second state;
determining, based at least in part on the first metric and the second metric, a third metric associated with the change in state of the portion of the human body; and
outputting the third metric.

2. The method of claim 1, wherein:
the portion of the human body is at least one foot;
the first state is in a non-load bearing state; and
the second state is in a load bearing state.

3. The method of claim 1, further comprising:
generating, based at least in part on the first image data, a first three-dimensional model of the portion of the human body; and
generating, based at least in part on the second image data, a second three-dimensional model of the portion of the human body.

4. The method of claim 1, wherein:
the portion of the human body is at least one foot;
the first metric is height of an arch of the foot in the non-load bearing state;
the second metric is a height of the arch of the foot in the load bearing state; and
the third metric is value representative of a navicular drop of an arch of the foot.

5. The method of claim 4, further comprising:
detecting a first ground plane associated with the first three-dimensional model;
filtering mesh points associated with the first three-dimensional model within a threshold distance from the first ground plane to generate a first set of remaining mesh points;
determining, based at least in part on the first set of remaining mesh points and the first ground plane, a first footprint associated with the foot in the non-load bearing state;
determining, based at least in part on the first three-dimensional model, a first convex hull associated with the foot in the non-load bearing state; and
determining, based at least in part on the first footprint and the first convex hull, a medial border line of the foot in the non-load bearing state.

6. The method of claim 5, further comprising:
detecting a second ground plane associated with the second three-dimensional model;
filtering mesh points associated with the second three-dimensional model within the threshold distance from the second ground plane to generate a second set of remaining mesh points;
determining, based at least in part on the second set of remaining mesh points and the second ground plane, a second footprint associated with the foot in the load bearing state;
determining, based at least in part on the second three-dimensional model, a second convex hull associated with the foot in the load bearing state; and
determining, based at least in part on the second footprint and the second convex hull, a medial border line of the foot in the load bearing state.

7. The method of claim 6, wherein detecting the first ground plane is associated with a first technique and detecting the second ground plane is associated with a second technique different than the first technique.

8. The method of claim 7, wherein:
determining the first footprint associated with the foot in the non-load bearing state further comprises projecting the first set of mesh points onto the first ground plane.

9. The method of claim 8, wherein determining the second footprint associated with the foot in the load bearing state further comprises projecting the second set of mesh points onto the second ground plane.

10. The method of claim 7, further comprising:
determining a first intersection point by projecting the medial border line of the foot in the non-load bearing state onto the first three-dimensional model;
determining a mid-point of the medial border line of the foot in the non-load bearing state;
determining a first distance between the first intersection point and the mid-point of the medial border line of the foot in the non-load bearing state, the first distance being the first metric;
determining a second intersection point by projecting the medial border line of the foot in the load bearing state onto the second three-dimensional model;
determining a mid-point of the medial border line of the foot in the load bearing state;
determining a second distance between the second intersection point and the mid-point of the medial border line of the foot in the load bearing state, the second distance being the second metric; and
determining a difference between the first distance and the second distance.

11. The method of claim 5, wherein the threshold distance is at least one of the following:
approximately 1.5 centimeters; or
approximately 1.0 centimeters.

12. The method of claim 1, wherein:
the portion of the human body is at least one foot;
determining the first metric associated with the foot while the foot is in the non-load bearing state is based at least in part on the first three-dimensional model; and
determining the second metric associated with the foot while the foot is in the load bearing state is based at least in part on the second three-dimensional model.

13. The method of claim 1, wherein the portion of the human body is at least one foot and generating the first three-dimensional model and the second three-dimensional model further comprises:
inputting the first image data into one or more machine learned models trained on image data of feet at various load bearing stages and receiving from the one or more machine learned models the first three-dimensional model; and
inputting the second image data into the one or more machine learned models and receiving from the one or more machine learned models the second three-dimensional model.

14. The method of claim 1, wherein the portion of the human body is at least one foot and determining the first metric, the second metric, or the third metric is further comprises utilizing one or more machine learning models trained on image data of feet in load bearing and non load bearing states of various different individuals having various lifestyles, health status, and demographics.

15. One or more non-transitory computer-readable media storing instructions that, when executed by one or more processors, cause the one or more processors to perform operations comprising:
receiving first image data of a foot in a non-load bearing state and second image data of the foot in a load bearing state;
determining, based at least in part on the first image data, a first height of an arch of the foot while the foot is in the non-load bearing state;
determining, based at least in part on the second image data, a second height of an arch of the foot while the foot is in the load bearing state;
determining, based at least in part on the first metric and the second metric, a third metric associated with the arch of the foot; and
outputting the third metric.

16. The one or more non-transitory computer-readable media of claim 15, wherein the third metric is a navicular drop of the arch of the foot.

17. A system comprising:
one or more processors; and
one or more non-transitory computer-readable media storing instructions that, when executed by the one or more processors, cause the one or more processors to perform operations comprising:
receiving first sensor data of a foot in a non-load bearing state and second sensor data of the foot in a load bearing state;
determining, based at least in part on the first sensor data, a first height of an arch of the foot while the foot is in the non-load bearing state;
determining, based at least in part on the second sensor data, a second height of an arch of the foot while the foot is in the load bearing state;
determining, based at least in part on the first metric and the second metric, a third metric associated with the arch of the foot; and
outputting the third metric.

18. The system of claim 17, wherein the operations further comprise:
determining the first metric associated with the arch of the foot is based at least in part on first-three dimensional model generated from the first image data; and
determining the second metric associated with the arch of the foot is based at least in part on second-three dimensional model generated from the second image data.

19. The system of claim 17, wherein:
the system is a cloud-based system that is remote from a user equipment; and
first sensor data and the second sensor data is received from the user equipment.

20. The system of claim 17, wherein the first sensor data and the second sensor data is one or more one of the following:
image data;
thermal data;
depth data;
infrared data;
magnetic resonance imaging data, or
LIDAR data.
